(19) United States
(12) Patent Application Publication
(10) Pub. No.: US 2005/0273043 A1
(43) Pub. Date: Dec. 8, 2005

(54) Tampon applicator providing low placement

(75) Inventors: Thomas Ward Osborn, III, Clifton, OH (US); Diana Lynne Gann, Lebanon, OH (US); Charles John Berg, JR., Wyoming, OH (US); Jacqueline Ann Daniels, Fairfield, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/861,760

(22) Filed: Jun. 4, 2004

(51) Int. Cl. A61F 13/20

(52) U.S. Cl. 604/15

(57) ABSTRACT

A tampon applicator for positioning a tampon inside a vaginal cavity. The tampon applicator includes an outer member disposed co-axially with an inner member for insertion of the tampon into the vaginal cavity. The inner member is slidable within the outer member wherein at least a portion of the tampon remains in the outer member when the inner member is fully engaged with the outer member to define a remaining portion of the tampon. The remaining portion of the tampon requires a force of less than about 0.30 N to be removed from the tampon applicator. Alternatively, when the inner member is fully engaged with the outer member to define a remaining portion of the tampon, the remaining portion of the tampon requires a force of less than about 0.04 N/mm to be removed from the outer member.

### FIELD OF INVENTION

This invention relates to a tampon applicator providing low placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and houses a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal and surrounding organs, further reducing the tampon coverage of menses flow in the vaginal cavity. Moreover, current tampon applicators are designed to “push” the tampon out of the outer tube substantially higher than the tip of the outer tube. This often causes the tampon to be deflected by the cervix/anterior fomix, resulting in an off-centered position of the tampon, and, thus, result in possible leakage from the vaginal cavity.

Alternatively, tampon applicators can place a tampon too low in the vaginal cavity. When the tampon is placed too low in the vaginal cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, it would be beneficial to provide a tampon applicator which properly places the tampon at a desired position in the vaginal cavity.

### SUMMARY OF THE INVENTION

The present invention encompasses a tampon applicator for positioning a tampon inside a vaginal cavity. The applicator includes an outer member disposed co-axially with an inner member for insertion of the tampon into the vaginal cavity. The inner member is slidable within the outer member wherein a portion of the tampon remains in the outer member when the inner member is fully engaged with the outer member to define a remaining portion of the tampon. The remaining portion of the tampon requires a force of less than about 0.30 N to be removed from the outer member.

In another embodiment herein, a tampon applicator includes an outer member disposed co-axially with an inner member for insertion of the tampon into the vaginal cavity. The inner member is slidable within the outer member. The outer member comprises an insertion end, an interior surface; and an exterior surface. The inner member comprises an interior surface, an exterior surface, a first end, and a second end. The tampon is at least partially housed within the inner member. A tampon positioning member is disposed within the inner member and co-axially with the tampon for expelling the tampon from the outer member wherein the tampon positioning member is joined to the interior surface of the inner member between the first end and the second end. In addition, a portion of the tampon remains in the inner member when the inner member is fully engaged with the outer member to define a remaining portion of the tampon. The remaining portion of the tampon requires a force of less than about 0.30 N to be removed from the inner member.

In another embodiment herein, a tampon applicator includes an outer member disposed co-axially with an inner member for insertion of the tampon into the vaginal cavity. The inner member is slidable within the outer member. The outer member comprises an insertion end, an interior surface, and an exterior surface. The inner member comprises an interior surface, an exterior surface, a first end, and a second end. The tampon is at least partially housed within the inner member. A tampon positioning member is disposed within the inner member and co-axially with the tampon for expelling the tampon from the outer member wherein the tampon positioning member is joined to the interior surface of the inner member between the first end and the second end. In addition, a portion of the tampon remains in the inner member when the inner member is fully engaged with the outer member to define a remaining portion of the tampon. The remaining portion of the tampon requires a force of less than about 0.30 N to be removed from the tampon applicator.

In another aspect, a tampon applicator includes an outer member disposed co-axially with an inner member for insertion of the tampon into the vaginal cavity. The inner member is slidable within the outer member. The outer member comprises an insertion end, an interior surface and an exterior surface. The inner member comprises an interior surface, an exterior surface, a first end, and a second end. The tampon is at least partially housed within the inner member. A tampon positioning member is disposed within the inner member and co-axially with the tampon for expelling the tampon from the outer member wherein the tampon positioning member is joined to the interior surface of the inner member between the first end and the second end. In addition, a portion of the tampon remains in the inner member when the inner member is fully engaged with the outer member by pushing the inner member into the outer member to define a remaining portion of the tampon. The remaining portion of the tampon requires a force of less than about 0.30 N to be removed from the inner member.

### BRIEF DESCRIPTION OF THE DRAWINGS

FIG. 1 is a perspective view along a longitudinal axis of a tampon applicator of the present invention.

FIG. 2 is a perspective view of the tampon applicator of the present invention.

FIG. 3 is a perspective view of the tampon applicator of the present invention when the tampon applicator is fully engaged.

FIG. 4 is a cross-sectional view along a longitudinal axis of the tampon applicator of the present invention.

FIG. 5 is a cross-sectional view along a longitudinal axis of the tampon applicator of the present invention.

FIG. 6 is a perspective view of an alternative embodiment of the tampon applicator of the present invention.

FIG. 7 is a cross-sectional view of an alternative embodiment of the tampon applicator of the present invention upon full engagement.

FIG. 8a is a perspective view of the Force Test Method equipment stand.

FIG. 8b is a perspective view of the Force Test Method equipment.

FIG. 8c is a perspective view of the Force Test Method equipment.

FIG. 9 is a graph of the results from the Force Test Method.

FIG. 10a is a graph of the results from the Force Test Method.

FIG. 10b is a graph of the results from the Force Test Method.

FIG. 10c is a graph of the results from the Force Test Method.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the tampon applicator of the present invention. Section C will discuss the Force Test Method.

A. Terms

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 mm long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 mm wide corresponding to the largest cylindrical cross section. The width can vary along the length of the self-sustaining tampon.

The second type of tampon is an easily “deformable, fluid-permeable bag tampon”. The deformable, fluid-permeable bag tampon consists of, but is not limited to, pieces such as absorbent chips, spheres, or fibers such that the fluid permeable bag tampon is readily deformable with a force of less than about 3 psi. The tampon is substantially deformable at pressures of less than about 1 psi; resulting in the tampon spreading or being easily indented when the pressure is applied from a surface of about 0.15 mm diameter.

As used herein, the terms “vaginal cavity” and “within the vagina” refer to the internal genitalia of the human female in the pudendal region of the body.

As used herein, the term “hymen ring” refers to the demarcation between the vaginal cavity and external genitalia and is identified by the position of the hymen or residual tissue of the hymen.

As used herein, the term “low placement” refers to a position of the tampon inside the vaginal cavity, wherein the base of the tampon is positioned at least about 5 millimeters above the hymen ring and below the cervix. The base of the tampon does not include any overwrap, secondary absorbent member, or withdrawal cord which extends beyond the tampon's main absorbent material.

As used herein, the term “remaining portion” refers to the portion of the tampon which is embedded into the outer member. Specifically, the remaining portion is measured from the most distal portion of the tampon to the most proximal portion of the outer member which includes petals or any extensions of the outer member.

As used herein, the term “proximal position” refers to the most proximal position of the tampon positioning member along the longitudinal axis.

The terms “distal” and “proximal,” respectively designated as P and D in the FIGS., herein respectively refer to directions away from and towards the body of the tampon wearer unless otherwise specified.

The term “fully engaged,” as used herein, occurs when the second end of the inner member is flush with the second end of the outer member or after 1250 grams of force has been applied to the end of the inner member.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Tampon Applicator of the Present Invention

The FIGS. show embodiments of the tampon applicator of the present invention. The present invention, however, is not limited to a structure having the particular configurations shown in the drawings or discussed herein. The tampon applicator of the present invention can be any configuration or size as long as the tampon can be removed from the tampon applicator by the body with a certain amount of force.

In addition to delivering menstrual tampons into the vaginal cavity, it should be noted that the tampon applicator of the present invention can be used to deliver any other type of absorbent or nonabsorbent object to any suitable cavity. For example, the tampon applicator of the present invention could be used to insert incontinence inserts. An “incontinence insert,” as used herein refers to devices specifically designed, configured, and/or adapted for placement into a vagina in order to reduce the occurrence and/or severity of female urinary incontinence. While incontinence inserts are typically made of non-absorbent materials, at least partially absorbent materials may also be used. However, because there is no intent to absorb bodily fluids, and because the incontinence inserts are adapted and configured to provide structural support to the musculature and body tissues located near the urethra-vaginal myfascial area, incontinence inserts are readily distinguishable from tampons.

In order to better understand the present invention, a detailed description of several non-limiting examples is given below. The below descriptions of the configurations of alternative embodiments are intended to be by way of example and are not intended to limit the invention.

I. Tampon Applicator Having an Outer Member and an Inner Member

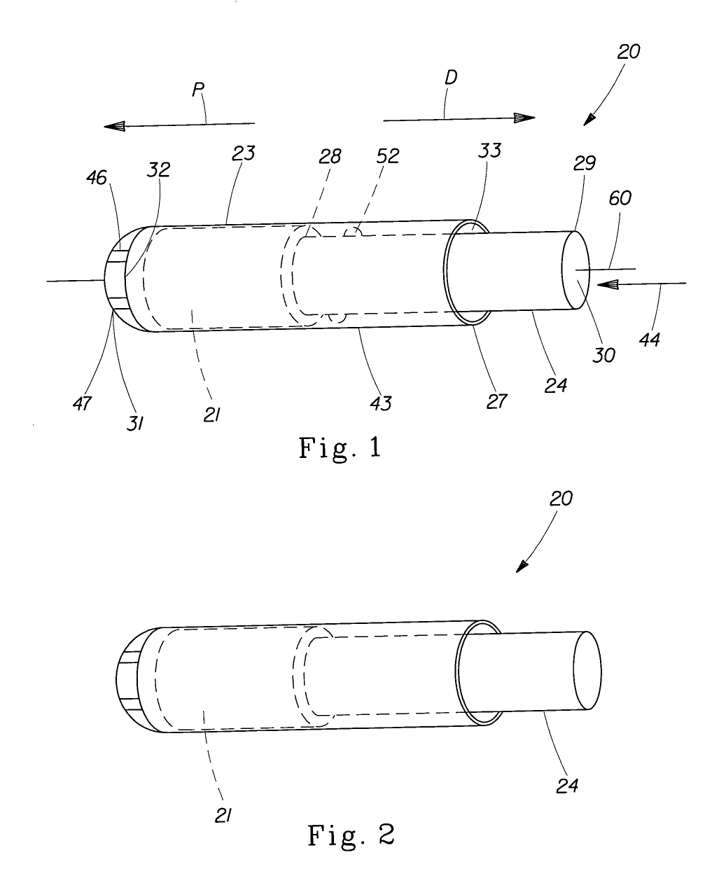

Referring to FIG. 1, a tampon applicator 20 is shown which is designed to position tampon 21 to achieve low placement inside the vaginal cavity. Specifically, the present invention is directed to a tampon applicator 20 which partially expels a tampon 21. Also, FIG. 1 shows “proximal” and “distal” designated as P and D, respectively. The tampon applicator 20 may be used with any type of tampon 21. The tampon 21 could be a self-sustaining tampon or a deformable fluid permeable bag tampon.

Generally, the tampon applicator 20 includes an outer member 23 and an inner member 24. The outer member 23 comprises an insertion end 31 and a second end 27 opposed to the insertion end 31. During insertion of the tampon applicator 20 into the body of a wearer, the insertion end 31 is the most proximal end to the body of the wearer along the longitudinal axis 60 and the second end 27 is the most distal end to the body of the wearer along the longitudinal axis 60. A preformed hinge or groove 32 may extend around the periphery of the outer member 23 near the insertion end 31. The outer member 23 may also have a dome-shaped end having a number of radial slits 46 therein extending from a central aperture to the groove 32. The portion of the outer member 23 adjacent to the insertion end 31 may also have an openable end, such as petals 47. In addition, the outer member 23 can contain a grip region 43 located adjacent to the second end 27 of the outer member 23.

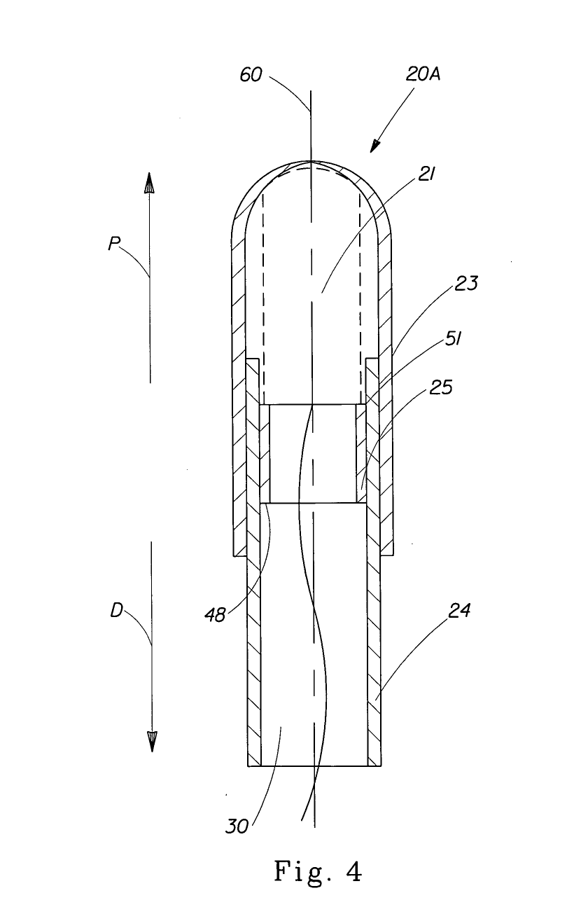

The inner member 24 is dimensioned to slidably move within the hollow interior portion 33 of the outer member 23, with minimal clearance therebetween. The inner member 24 has a first end 28 and a second end 29 opposed to the first end 28. The first end 28 is the most proximal end of the inner member 24 along the longitudinal axis 60. In this embodiment, the first end 28 provides the necessary force to expel the exposed portion 35 (FIG. 3) of tampon 21. In other words, the first end 28 becomes the tampon pushing member 51. The inner member 24 may have nibs 52 located near the first end 28 of inner member 24. The second end 29 is the most distal end of the inner member 24 along the longitudinal axis 60. An axial force 44 is applied to the second end 29 of inner member 24 to expel the exposed portion 35 (FIG. 3) of tampon 21. Also, the inner member 24 can have a hollow interior portion 30. Alternatively, the inner member 24 can be solid or partially solid.

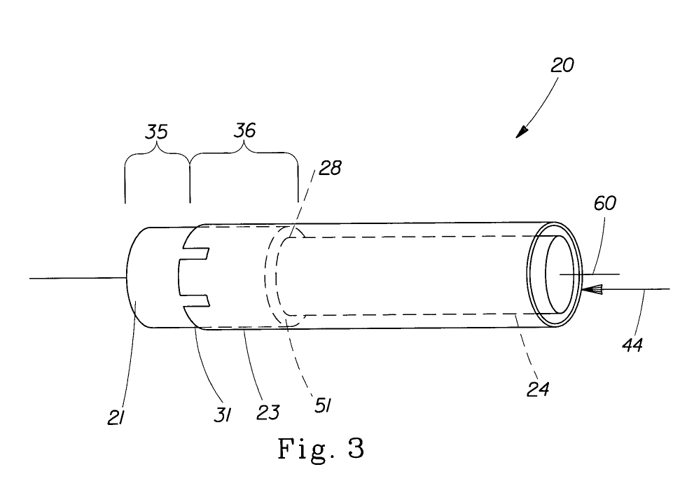

The tampon applicator 20 has a pre-expelled state and a partially expelled state. During the pre-expelled state, as is readily seen in FIG. 1, the tampon 21 sits within the outer member 23 and is substantially aligned with the tampon applicator 20. The tampon 21 can remain snugly therein without any outside force to sustain its position in the tampon applicator 20. In one nonlimiting example, the tampon 21 can be enveloped by the inner member 24 or embedded within inner member 24 during expulsion of tampon 21. The tampon 21 may or may not be in contact with the first end 28 of the inner member 24 before expulsion of the tampon 21. In one nonlimiting example, as seen in FIG. 2, before expulsion of the exposed portion 35 (FIG. 3) of tampon 21 from the tampon applicator 20, the tampon 21 does not contact the inner member 24 and can be substantially aligned with the tampon applicator 20.

As is readily seen in FIG. 3, when the axial force 44 is applied along the longitudinal axis 60, the inner member 24 slides toward the insertion end 31 of the outer member 23. The inner member 24 bears against the rear end of tampon 21 pushing the tampon 21 toward the insertion end 31 of outer member 23. In this embodiment, the first end 28 becomes the tampon pushing member 51. The tampon pushing member 51 provides the necessary force to expel the exposed portion 35 of tampon 21. Upon full engagement of inner member 24 with outer member 23, the exposed portion 35 of the tampon 21 is expelled from the tampon applicator 20 by the tampon pushing member 51 and a remaining portion 36 of the tampon 21 is contained within outer member 23.

During removal of the tampon applicator 20 from the body cavity, the body tissues hold onto the exposed portion 35 of the tampon 21 with the adherence of the absorbent tampon surface to vaginal tissues and body pressure. Because the body tissues hold onto the exposed portion 35 of the tampon 21, the tampon applicator 20 exits the body cavity without the tampon 21, i.e., leaving the tampon 21 behind. This results in low placement of the tampon 21 within the vagina.

II. Tampon Applicator Having a Tampon Positioning Member

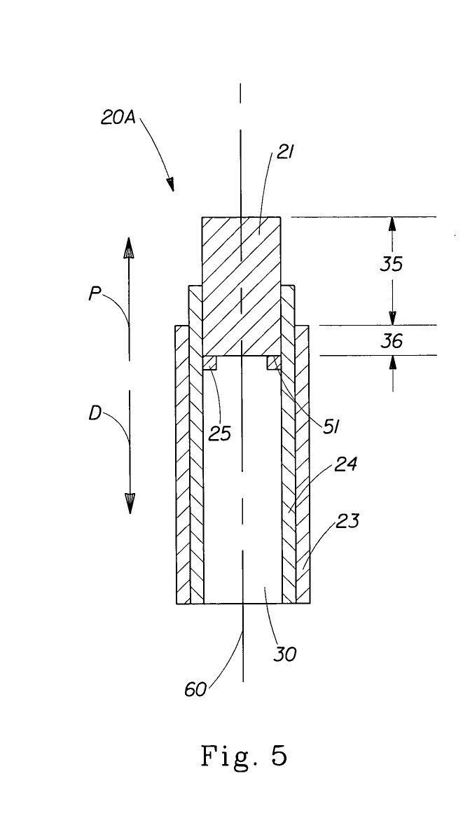

Referring to FIG. 4, in another alternative embodiment, a tampon applicator 20A is shown which is designed to position tampon 21 to achieve low placement inside the vaginal cavity. Also, FIG. 4 shows “proximal” and “distal” designated as P and D, respectively. Generally, the tampon applicator 20A comprises an outer member 23 and an inner member 24 as discussed in the below section. In addition, the hollow interior 30 of the outer member 23 may contain a tampon positioning member 25.

Referring again to FIG. 4, the tampon positioning member 25 can have a tampon pushing member 51 and a second end 48 opposed to the tampon pushing member 51. The tampon pushing member 51 is the most proximal end of the tampon positioning member 25 along the longitudinal axis 60. The tampon pushing member 51 contacts the tampon 21 and positions the tampon 21 along the length of the inner member 24 so that during expulsion the tampon pushing member 51 forces the tampon 21 to travel a pre-determined distance. This predetermined distance ultimately results in the tampon 21 being positioned low in the vaginal cavity. The second end 48 is the most distal end of the tampon positioning member 25 along the longitudinal axis 60. In one non-limiting example, the tampon positioning member is a flat surface.

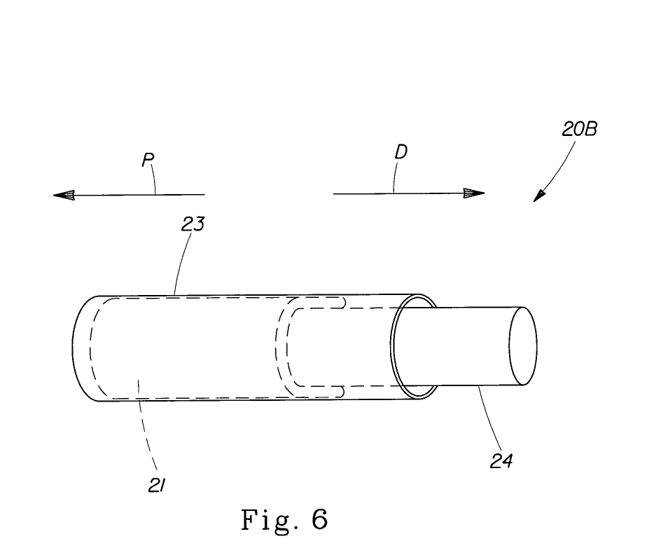

Referring to FIG. 5, in this embodiment, during expulsion of the exposed portion 35 of the tampon 21 from tampon applicator 20A, the remaining portion 36 is embedded in the outer member 23 when the tampon pushing member 51 of the tampon positioning member 25 is in its proximal position and when the tampon applicator 20A has full engagement by having the inner member 24 flush with the outer member 23. Also, FIG. 5 shows “proximal” and “distal” designated as P and D, respectively.

During full engagement of tampon applicator 20A, the tampon pushing member 51 positions tampon 21 such that tampon 21 has an exposed portion 35 expelled from tampon applicator 20A and a remaining portion 36 housed within the inner member 24. During expulsion of the exposed portion 35 of tampon 21 from the tampon applicator 20A, at least a portion of the tampon 21 is adjacent to the tampon pushing member 51 of the tampon positioning member 25. In other words, when the tampon pushing member 51 is at its proximal position along longitudinal axis 60, the tampon pushing member 51 is in contact with the tampon 21 and provides the force necessary to expel the exposed portion 35 of the tampon 21. The body forces inside of the vagina hold onto the exposed portion 35 of the tampon 21 during removal of the tampon applicator 20A from the vaginal cavity.

The tampon positioning member 25 can be fixedly joined to the inner member 24 in any way known in the art. The tampon positioning member 25 can also be monolithic with the inner member 24, for example, by molding the tampon positioning member 25 and the inner member 24 as one piece. In one non-limiting example, the tampon positioning member 25 can be fixedly joined to the inner member 24 by friction fitting that snaps parts together, gluing, and/or melting. In another non-limiting example, the tampon positioning member 25 is attached to the hollow interior portion 30 of the inner member 24. In another non-limiting example, the tampon positioning member 25 can also be a continuation of the inner member 24 by folding or compressing a portion of the inner member 24 and turning the inner member 24 inwards upon itself to create a tube inside a tube.

It is further noted herein that the shape of the tampon positioning member 25 can vary as long as the tampon positioning member 25 aids in positioning the tampon 21 along the length of the inner member 24 to provide low placement of the tampon 21 within the vaginal cavity. In one embodiment, the tampon positioning member includes an elongated tampon positioning member of a generally tubular shape. Alternatively, the tampon positioning member can be a rod. In another embodiment, the tampon positioning member can be slideable within the outer member 23. In another alternative embodiment, the tampon positioning member can also comprise projections extending inward from the hollow interior 30 of the inner member 24. The projections can be of any shape or size as long as the projections allow the tampon 21 to be in contact with the projections during the expulsion. The projections can be fixedly joined to the inner member 24 in any way known in the art.

It is further noted herein that the length of the tampon positioning member 25 can vary as long as the tampon positioning member 25 aids the tampon 21 to be located in a position within the inner member 24 that will provide low placement within the vaginal cavity. In one non-limiting example, the length of the tampon positioning member 25 can be from about 36 millimeters to about 63 millimeters.

III. Inner Member Embedded into Tampon

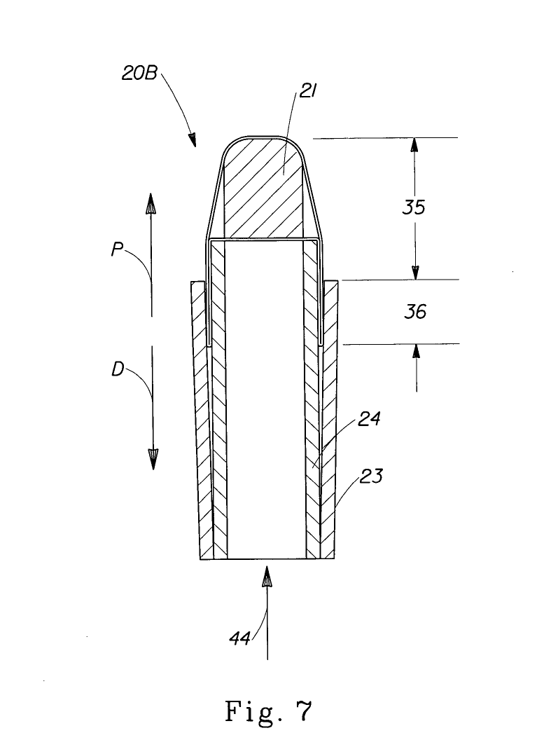

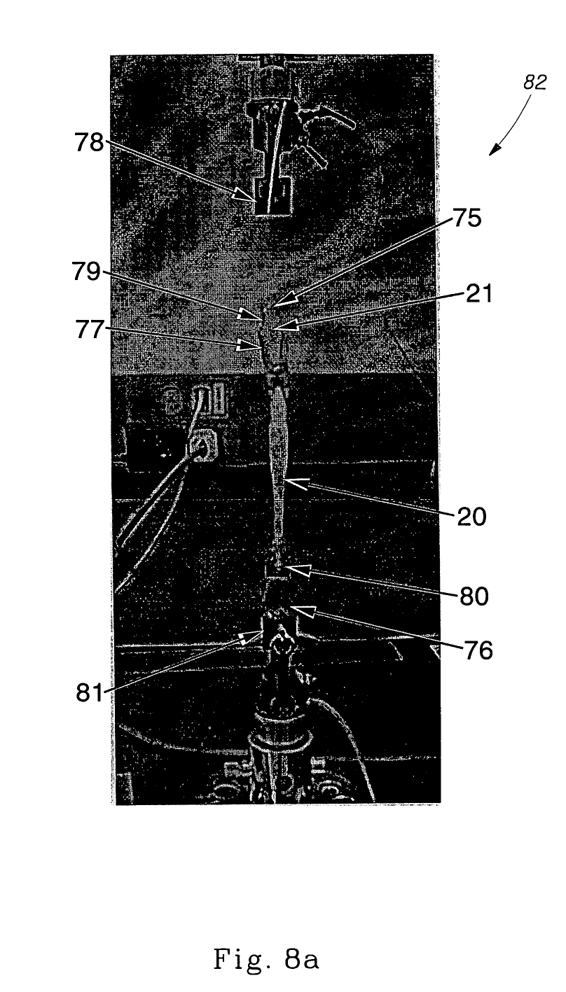

Referring to FIG. 6, in yet another alternative embodiment, a tampon applicator 20B is shown before expulsion of the exposed portion 35 (FIG. 7) of tampon 21. Tampon applicator 20B is designed to position tampon 21 to achieve low placement inside the vaginal cavity. Also, FIG. 6 shows “proximal” and “distal” designated as P and D, respectively. In this non-limiting example, generally, the tampon applicator 20B comprises an outer member 23 and an inner member 24. In this alternative embodiment, however, the inner member 24 can be embedded within the tampon 21 upon full engagement of the tampon applicator 20B. In other words, the inner member 24 can penetrate at least a portion of the base of the tampon 21 upon full engagement of the tampon applicator 20B.

Referring to FIG. 7, upon full engagement of the outer member 23 with the inner member 24, an exposed portion 35 is expelled from the tampon applicator 20B and a remaining portion 36 of the tampon 21 is contained within tampon applicator 20B. Also, FIG. 7 shows “proximal” and “distal” designated as P and D, respectively. In addition, upon full engagement at least a portion of the inner member 24 is adjacent to the tampon 21. When the inner member 24 is embedded into tampon 21, the inner member 24 provides the force 44 to expel the exposed portion 35 of tampon 21 from tampon applicator 20B.

IV Examples of the Tampon Applicator of the Present Invention

Examples 1-13, provided below, are embodiments of the tampon applicator of the present invention which requires a certain amount of force to remove the remaining portion of the tampon from the applicator upon full engagement during removal of the tampon applicator from the vaginal cavity. The present invention is not limited to the above structures having the particular configurations shown in the drawings or discussed above herein. Thus, the tampon applicator can be any configuration or size.

| | Force(N) Required to |
|---|---|
| | Remove the Remaining |
| | Portion of the Tampon from |
| | the Tampon Applicator upon |
| Example | Full Engagement |
| Example 1 | Less than about .30 |
| Example 2 | Less than about .25 |
| Example 3 | Less than about .20 |
| Example 4 | Less than about .15 |
| Example 5 | Less than about .10 |
| Example 6 | Less than about .26 |
| Example 7 | Less than about .27 |
| Example 8 | Less than about .50 |
| Example 9 | Less than about .40 |
| Example 10 | Less than about .28 |
| Example 11 | Less than about .18 |
| Example 12 | Less than about .48 |
| Example 13 | Less than about .46 |

Examples 14-20, provided below, are embodiments of the tampon applicator of the present invention which requires a certain amount of force to remove the remaining portion of the tampon from the applicator upon full engagement during removal of the tampon applicator from the vaginal cavity. The present invention is not limited to the above structures having the particular configurations shown in the drawings or discussed above herein. Thus, the tampon applicator can be any configuration or size.

| | Force(N) Required to |
|---|---|
| | Remove the Remaining |
| | Portion of the Tampon from |
| | the Tampon Applicator upon |
| Example | Full Engagement |
| Example 14 | Less than about 0.50 |
| Example 15 | Less than about 0.48 |
| Example 16 | Less than about 0.46 |
| Example 17 | Less than about 0.35 |
| Example 18 | Less than about 0.33 |
| Example 19 | Less than about 0.30 |
| Example 20 | Less than about 0.40 |

Examples 21-24, provided below, are embodiments of the tampon applicator of the present invention which expels an exposed portion of the tampon upon full engagement of the tampon applicator of the present invention. The present invention requires a certain amount of force per millimeter to remove the remaining portion of the tampon from the tampon applicator of the present invention. The present invention, however, is not limited to the above structures having the particular configurations shown in the drawings or discussed above herein. Thus, the tampon applicator can be any configuration or size.

| | Force(N/mm) Required to |
|---|---|
| | Remove the Remaining |
| | Portion (i.e. Embedded |
| | Portion) of the Tampon from |
| | the Tampon Applicator upon |
| Example | Full Engagement |
| Example 21 | Less than about 0.04 |
| Example 22 | Less than about 0.03 |
| Example 23 | Less than about 0.02 |
| Example 24 | Less than about 0.01 |

Examples 25-26 provided below, are embodiments of the tampon applicator of the present invention in which a percentage of the tampon remains embedded within the outer member upon full engagement of the inner member with the outer member. Also, a certain amount of force per millimeter is required to remove the remaining portion of the tampon from the tampon applicator during removal of the tampon applicator from the vaginal cavity. The present invention is not limited to the above structures having the particular configurations shown in the drawings or discussed above herein. Thus, the tampon applicator can be any configuration or size.

| | Force(N) Required to |
|---|---|
| | Remove the Remaining |
| | Portion of the Tampon from |
| | the Tampon Applicator upon |
| Example | Full Engagement |
| Example 25 | Less than about 0.04 |
| Example 26 | Less than about 0.03 |

Below will provide more detail of each component of the tampon applicator of the present invention.

a. Outer Member

Referring to FIG. 1, typically, the outer member 23 can be used to handle or grip the tampon applicator 20 during the insertion into the vaginal cavity. The outer member 23 is external to the inner member 24. The outer member 23 has a hollow interior 33, an insertion end 31, and a second end 27 opposed to the insertion end 31. The insertion end 31 is the most proximal end of the outer member 23. In addition, the insertion end 31 is the first portion of the outer member 23 which is inserted into the vaginal cavity and it is the end from which the tampon 21 is expelled. The second end 27 is the most distal portion of the outer member 23 along the longitudinal axis 60. Grip region 43 is adjacent to the second end 27. Grip region 43 is the portion of the outer member 23 in which the user can handle or grip the tampon applicator 20. At least a portion of the hollow interior 33 of the outer member 23 can engage with at least a portion of the inner member 24. In one non-limiting example, the inner member 24 can be slideable within the outer member 23.

The manufacturer of the tampon applicator 20 can vary the size of the outer member 23. The size of the outer member 23 can be determined primarily by the dimensions of the tampon 21. Specifically, the diameter of the outer member 23 can be varied to accommodate different absorbency tampons. Generally, the outer member 23 can have an inner diameter of from about 6 millimeters to about 21 millimeters and a wall thickness of from about 0.4 millimeter to about 1.2 millimeter. Generally, the inner diameter of the outer member 23 should be suitably greater than the diameter of the tampon 21 to prevent the outer member 23 from interfering with the removal of the tampon 21 from the outer member 23. Moreover, the inner diameter of the outer member 23 can have varying diameters and shapes to conform to the profiled shape of the enclosed tampon 21.

Also, the manufacturer of the tampon applicator 20 can vary the length of the outer member 23. Generally, the outer member 23 should be of a sufficient length to house at least a portion of the tampon 21 and preferably all of tampon 21 prior to the expulsion of the tampon 21 from the tampon applicator 20 into the vaginal cavity. Generally, the length of the outer member 23 can be from about 50 millimeters to about 100 millimeters. The length of the outer member 23 is preferably from about 50 mm to about 90 mm, more preferably from about 55 mm to about 85 mm.

In addition, the manufacturer of the tampon applicator 20 can vary the shape of the outer member 23. It is further noted herein that the shape of the outer member 23 can vary as long as a portion of the inner member 24 can be slideable within the outer member 23. In one non-limiting example, the grip region 43 can be of a smaller perimeter than the remainder of the outer member 23. In this non-limiting example, the inner member 24 can be a rod which slidably moves within the hollow interior portion 33 of the outer member 23. Moreover, the outer member 23 is in no way limited by the shape that it can assume except that the shape should not hinder directional expulsion of the tampon 21. One skilled in the art can imagine that the outer member 23 might be cylindrical or curved like a banana or any other suitable shape as long as any such shape would work effectively to allow tampon 21 to expel from the tampon applicator 20, and comfortably insert into the vaginal cavity. The outer member 23 can be of any suitable cross-sectional shape. For example, suitable cross-sectional shapes can include, but are not limited to, circular, oval, flattened circular, elliptical, and any combination thereof.

Furthermore, the outer member 23 can contain the grip region 43 as noted above. The grip region 43 is located between the insertion end 31 and the second end 27 and closer to the second end 27 of the outer member 23. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. Referring again to FIG. 1, the grip region 43 is substantially circularly shaped, but it can also take on more angular formations such as squared. The surface of the grip region 43 can be any kind of surface known in the art. This surface can provide a desired frictional resistance for the fingers during the insertion of the tampon applicator 20 into the body. Moreover, this surface area can have, for example, a dove pattern created via surface texturing, impressions, and/or indentations.

Furthermore, it is preferable that the grip region 43 be of sufficient dimension to substantially provide a comfortable grip for the user. As used in the specification, the term “user's grip” means any way of holding the tampon applicator 20 in a hand, e.g., between a thumb and a finger. The grip region 43 need not necessarily extend completely around the perimeter of the outer member 23. For example, a space can be provided for a decorative marking or a Trademark character.

b. Inner Member

In FIG. 1, in some embodiments of the present invention, inner member 24 can be designed to store at least a portion of the tampon 21 within its hollow interior 30 either during the expulsion and/or before the expulsion of the tampon 21 from the insertion end 26 of the outer member 23. The inner member 24 has a hollow interior 30. Alternatively, the inner member 24 can be solid or partially solid. The first end 28 is the most proximal end of the inner member 24 along the longitudinal axis 60. The second end 29 is the most distal end of the tampon applicator 20 along the longitudinal axis 60. Moreover, the second end 29 is the portion of the inner member 24 in which the axial force 44 is applied to expel the tampon 21 from the outer member 23.

The manufacturer of the tampon applicator 20 can vary the shape of the inner member 24 as long as any such shape would work effectively to properly aide in the expulsion of the tampon 21 from the tampon applicator 20 and comfortably insert into the vaginal cavity. One skilled in the art can imagine other shapes of the inner member 24, for example, cylindrical or curved like a banana or any other suitable shape possible. For example, the inner member 24 can be of any suitable cross-sectional shape including, but are not limited to, circular, oval, flattened circular, elliptical, and any combinations thereof.

The size of the inner member 24 can be determined primarily by the dimensions of tampon 21. Specifically, the diameter of the inner member 24 can be varied to accommodate different absorbency tampons. For example, when tampon 21 is housed within the inner member 24, higher absorbency tampons can have larger diameters resulting in the inner diameter of the inner member 24 having a larger diameter to house the higher absorbent tampon. In addition, the inner diameter of the inner member 24 can have varying diameters and shapes to conform to the profiled shape of the enclosed tampon 21.

The length of the inner member 24 can also vary. If the tampon 21 is housed within inner member 24, generally, the inner member 24 should be of a sufficient length to house a substantial portion of the tampon 21 prior to the insertion of the applicator into the body, thus aiding in the proper expulsion of the tampon 21 from the tampon applicator 20, and comfortably inserting into the vaginal cavity. For example, the length of the inner member 24 can be from about 45 millimeters to about 90 millimeters.

Optionally, it can be seen that inner member 24 can have nibs 52 provided on the perimeter thereof located near the first end 28 of inner member 24. If nibs 52 are included, the inner member 24 should fit within the outer member 23. The nibs 52 can be adapted to engage with the optional grooves on the inside surface (not shown) of outer member 23 thereby providing a number of possible predetermined relative positions for the outer member 23 and for the inner member 24. Nibs 52 can prevent the inner member 24 from too easily being pushed into the outer member 23 (resulting in premature expulsion of the tampon 21) should the tampon applicator 20 be mishandled before the use thereof. Nibs may also be used to stop the movement of the inner member 24 to signal full engagement of the inner member 24 and the outer member 23.

c. Tampon Applicator Materials

Different tampon applicator parts can be constructed from different materials and processes. The tampon applicator or any part of the tampon applicator can be formed of a spirally wound, convolutedly wound, or longitudinally seamed hollow tube that is formed from paper, paperboard, cardboard, or any combinations thereof. The tampon applicator or any part of the tampon applicator can also be injection molded, extruded, or formed from flexible plastic, such as thermoformed from plastic sheet or folded or wound from plastic film.

The tampon applicator or any part of the tampon applicator can be constructed from a single ply of material or be formed from two or more plies that are bonded together to form a laminate. The use of two or more plies or layers is preferred for it enables the manufacturer to use certain materials in the various layers that can enhance the performance of the tampon applicator or any part of the tampon applicator. When two or more plies are utilized, all the plies can be spirally wound, convolutedly wound, or longitudinally seamed to form an elongated cylinder. The tampon applicator or any part of the tampon applicator can be constructed using a smooth thin ply of material on the outside or exterior surface that surrounds a coarser and possibly thicker ply. When the tampon applicator or any part of the tampon applicator contains at least three plies, the middle ply can be the thicker ply, and the interior and exterior plies can be smooth and/or slippery to facilitate the expulsion of the tampon and to facilitate the insertion of the tampon applicator or any part of the tampon applicator into a woman's vagina. By sandwiching a thick, coarser ply of material between two thin, smooth plies, a tampon applicator or any part of the tampon applicator can be provided which is very functional. The tampon applicator or any part of the tampon applicator should contain one to four plies, although more plies can be utilized if desired.

The plies forming the tampon applicator or any part of the tampon applicator can be held together by an adhesive, such as glue, heat, pressure, ultrasonic, or any combinations thereof. The adhesive can be either water-soluble or water-insoluble. A water-soluble adhesive is preferred for environmental reasons in that the tampon applicator or any part of the tampon applicator will quickly break apart when it is immersed in water. Such immersion will occur should the tampon applicator or any part of the tampon applicator be disposed of by flushing it down a toilet. Exposure of the tampon applicator or any part of the tampon applicator to a municipal's waste treatment plant wherein soaking in water, interaction with chemicals, and agitation all occur, will cause the tampon applicator or any part of the tampon applicator to break apart and evenly disperse in a relatively short period of time.

C. Force Test Method

i. Overview

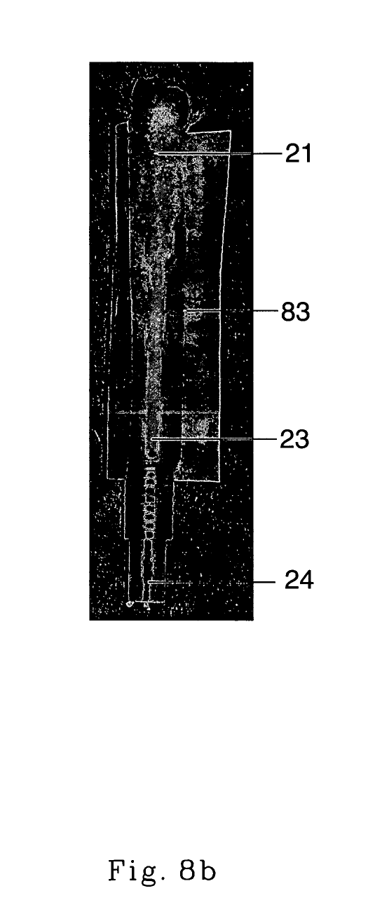

Referring to FIG. 8a, this test is intended to measure the amount of force that is required to remove the remaining portion of the tampon from a tampon applicator which is fully engaged.

The less length of the tampon 21 exposed after expulsion from the tampon applicator, the lower the tampon 21 is placed in the vaginal cavity when compared to an identical tampon-applicator system where the tampon is fully expelled. When the tampon 21 is placed low in the vaginal cavity there is greater coverage in the vaginal cavity because a tampon 21 placed lower in the vagina is more effective in covering the vagina given that the vagina is typically narrower in the lower region and the tampon is placed in the center and front of the cervix.

ii. Test Stand Preparation

The Force Removal Test uses an Instron Model 5564 with a 10 Newton load cell, which is available from Instron Corporation of Canton, Mass.; standard 1.0 inch grips operated at 60 psi were used. The test stand also uses two ¾″ coated plastic tip mini-Spring Clamps (model number 614-593 WorkForce, 2455 Paces Ferry Rd, Atlanta, Ga. 30339 purchased from Home Depot (#3579462622 8)); 0.0198 inch diameter fishing line manufactured by Shakespeare Model WP550; and a folded wire paper clip (no. 1 Gem Clip, Model XP40020).

Machinery Settings: The Instron crosshead speed is set at 508 mm/min. The force on the fishing line 79 is adjusted to +/−5 grams so that it sets a standard initial force on the tampon 21 and eliminates significant excess slack in the fishing line 79. The cross head travel is the distance required for the tampon 21 to be fully removed from the tampon applicator 20. Data is collected at 100 Hz starting at 2 mm of cross head extension. The peak force of the test is the maximum force in Newtons obtained as the tampon 21 is removed from the tampon applicator 20 minus the Baseline Force contributed by the test tampon, upper utility clip assembly, and fishing line. The Baseline Force may be determined by the force applied to the Instron after the tampon 21 is totally removed from the tampon applicator. Ten tampon-applicator assemblies are analyzed and the average force is reported.

The following steps occur to prepare the test stand 82 as shown in FIG. 8a:

1. Gem Clips: Unfold the Gem Clips 75, 76 until the Gem Clips 75, 76 form a substantially straight line. Form the clips into a “U” or “V” shape.

2. Upper Utility Clip Assembly: Run both ends of the first Gem Clip 75 through the holes at the base of the upper mini-Spring Clamp 77 so that the U portion of the Gem Clip 75 is facing away from the mini-Spring Clamp 77. The Gem Clip 75 is secured to the mini-Spring Clamp 77 by bending and twisting it around itself. Secure a first end of the 12 inch section of fishing line 79 to the top of the center U of the first Gem Clip 75.

3. Lower Utility Clip Assembly: Run the ends of the second Gem clip 76 through the holes at the base of lower mini-Spring Clamp 80. Form the second Gem paper clip 76 into a substantially U-shaped configuration and secure the second Gem clip 76 to the mini-Spring Clamp 77 by bending and twisting it around itself.

4. Prepare Stabilizing Fixture. Referring to FIG. 8b, a Stabilizing Fixture 83 may be required to stabilize the fully plunged tampon-applicator system 20 as it is placed into the test stand 82 (FIG. 8a). The stabilizing fixture 83 is a flexible partial tube made from paper manila letter size File Folders obtained from The Smead Manufacturing Co., Hastings, Mn. Part number 135L, UPC No. 10330. The paper folder is sized such that when the inner member 24 is fully engaged the tampon extends about 1 cm from the longitudinal edge and its width is from about 1 mm to about 5 mm less than the outer member 23 of the tampon applicator 20. The paper is then rolled along the longitudinal axis into a partial tube that can be used to hold the tampon-applicator assembly together. While the Stabilizing Fixture 83 may be adjusted to the specific dimensions of the tampon applicator 20, typical dimensions are about 85 mm long and about 50 mm in width prior to forming the tube.

ii. Sample Preparation

The Force Test is performed on tampon applicator samples and the present tampon applicator. The following are the tampon applicator samples:

Walgreens® Plastic super plus absorbency
Walgreens® Plastic Tampons super absorbency
Walgreens® Plastic Tampons regular absorbency
Playtex® Gentle Glide super absorbency labeled incredible comfort and protection
Playtex® Gentle Glide regular absorbency labeled incredible comfort and protection
Playtex® Slimfits regular absorbency
Unicharm (Sofy Soft Tampon)
Test Sample 1 Regular Absorbency
Test Sample 2 Regular Absorbency

To prepare the sample tampon for use in the Force Test Method, the withdrawal string of the tampon must be trimmed so that it is flush with the inner member. The withdrawal string is trimmed to prevent it from catching a surface when the tampon is extracted from the tampon applicator during any inner member movement, or during the Instron test.

Referring to FIG. 8a, the following must be done to prepare the samples prior to testing:

1. The tampon applicator samples are removed from the box and equilibrated at 23° C.±1° C. and at a relative humidity of 50%±2% for 2 hours within the tampon applicator and wrapper, if tampons are individually wrapped.

2. Remove the tampon from the wrapper (if present) and complete the sample preparation and testing within 10 minutes after removal from the wrapper.

3. Determination of Plunge length. Plunge the tampon applicator 20 by pushing the inner member 24 against a solid flat surface until the inner member is fully engaged. A full inner member 24 engagement with outer member 23 is completed when 1250 g of force is applied to the inner member 24. If the tampon 21 is not retained in the tampon applicator 20 or retained by less than 2 mm, the sample is outside the limits of this test as determined by the Force Test Method.

a. Place the tampon applicator assembly horizontally onto the Stabilizing Fixture 83 so that when the tampon 21 is deployed the leading edge of the tampon extends about 1 cm from longitudinal edge of one end of the Stabilizing Fixture 83. The grip zone of the applicator 20 should extend about 1 cm from the opposite end of the Stabilizing Fixture 83.

b. Grasp the grip zone of the tampon applicator 20 and plunge the tampon applicator 20 to the fully engaged position.

c. Carefully grasp the side of the Stabilizing Fixture 83 about 1 cm below the proximal end of the tampon applicator 20, applying only sufficient pressure to hold the tampon-applicator system 20 in a stable configuration.

d. Referring to FIG. 8c, attach the Upper Utility Clip Assembly 77 to the top 1 cm of the proximal end of the tampon 21.

e. Referring to FIG. 8a, attach the Lower Utility Clip Assembly 80 to the finger grip of the tampon applicator 20. While carefully holding the tampon-applicator system in the Stabilizing Fixture 83 (FIG. 8b), seat the concave portion of the substantially u-shaped configuration of the GEM clip of the Lower Utility Clip Assembly 76 into the lower Instron grip 81. This will allow the Lower Utility Clip Assembly 76 to be anchored in the lower Instron grip 81 of the test stand 82.

f. Insert the fishing line of the Upper Utility Clip Assembly 77 into the upper Instron grip 78. Carefully remove the Stabilizing Fixture 83 (FIG. 8b) from around the tampon-applicator assembly.

g. If the tampon is displaced or misaligned during the assembly or during the attachment between the Instron grips 81, discard the sample and repeat the procedure.

iii. Results from Force Test

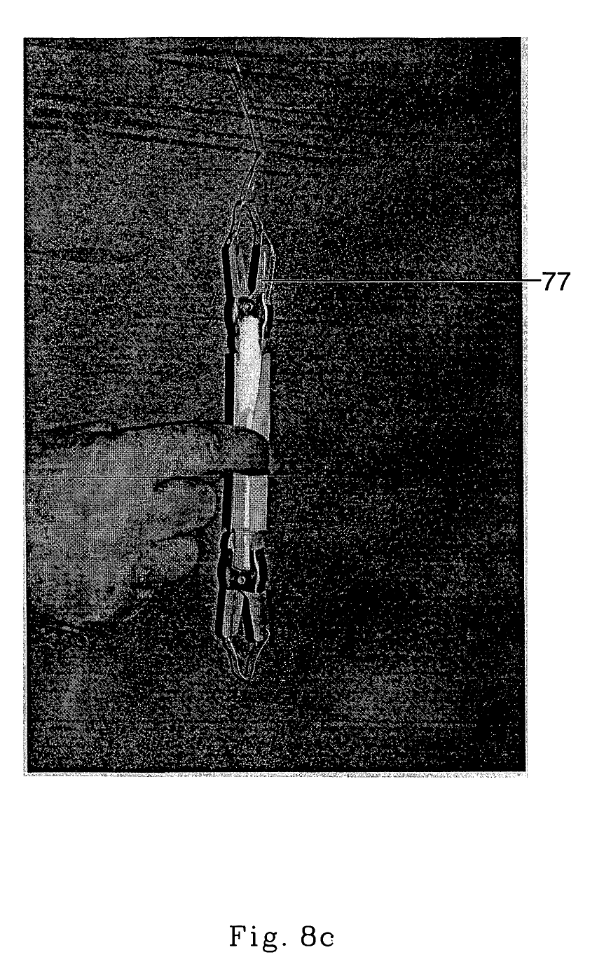

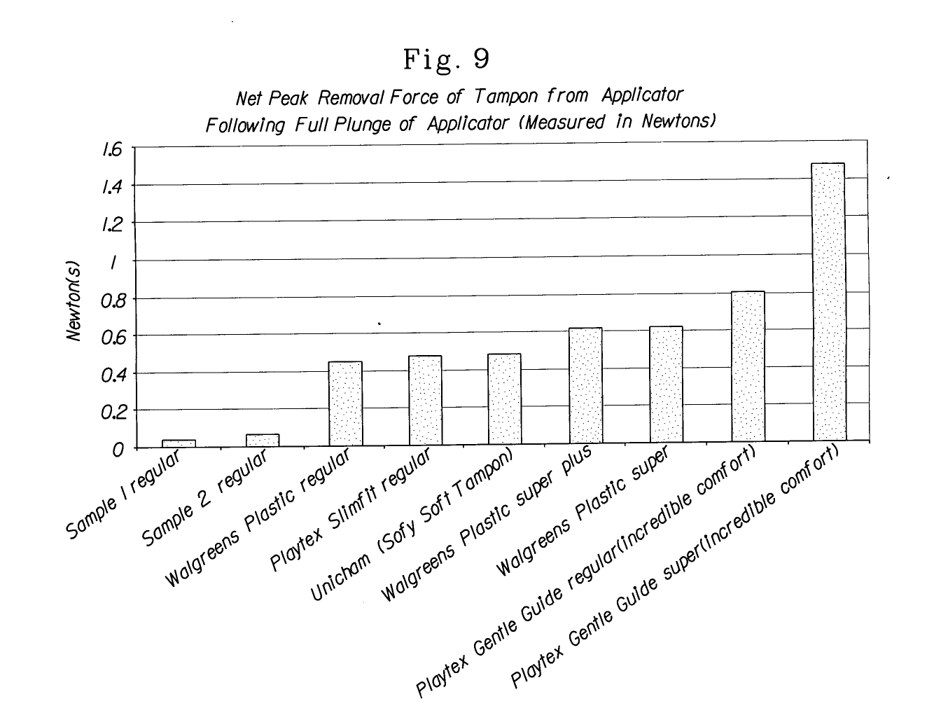

Referring to FIGS. 9 and 10a, the test reveals that the Test Sample 1 and Sample 2 of the tampon applicators of the present invention require less peak force as measured in Newtons and Newtons/mm to remove the tampon from the tampon applicator than applicators of the current art. Referring to FIG. 10b, when using the Force Test Method disclosed herein, five samples of Playtex® Gentle Glide super absorbency labeled incredible comfort and protection will have an average net peak load measured in Newtons of 0.207. Referring to FIG. 10c, when using the Force Test Method disclosed herein, samples of the present invention will have an average net peak load measured in Newtons of 0.036. The clip, line, & tampon weight are subtracted from the net peak load. During removal of the tampon applicator from the body cavity, less force is required to remove the tampon from the tampon applicator 21 so that body tissues can hold onto the tampon 21 due to the adherence of the absorbent surface to vaginal tissues and due to body pressure. Since the tampon is not fully expelled from the tampon applicator, the body tissues are required to hold onto the tampon 21, extracting it from the tampon applicator as it exits the body cavity without the tampon 21, i.e., leaving the tampon 21 behind. This results in low placement of the tampon 21 within the vagina compared to tampons fully expelled from applicators of similar length.

All documents cited in the Detailed Description of the Invention are incorporated, in relevant part, herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A tampon applicator for positioning a tampon inside a vaginal cavity, said applicator comprising:
i. an outer member disposed co-axially with an inner member for insertion of said tampon into said vaginal cavity;
ii. said inner member being slidable within said outer member
wherein a portion of said tampon remains in said outer member when said inner member is fully engaged with said outer member to define a remaining portion of said tampon, said remaining portion of said tampon requiring a force of less than about 0.30 N to be removed from said outer member.

2. The tampon applicator according to claim 1 wherein said remaining portion of said tampon requires a force of less than about 0.25 N to be removed from said outer member.

3. The tampon applicator according to claim 1 wherein said remaining portion of said tampon requires a force of less than about 0.20 N to be removed from said outer member.

4. The tampon applicator according to claim 1 wherein said remaining portion of said tampon requires a force of less than about 0.15 N to be removed from said outer member.

5. A tampon applicator for positioning a tampon inside a vaginal cavity, said applicator comprising:
i. an outer member disposed co-axially with an inner member for insertion of said tampon into said vaginal cavity, said inner member being slidable within said outer member, said outer member comprising an insertion end, an interior surface and an exterior surface, said inner member comprising an interior surface, an exterior surface, a first end and a second end;
ii. said tampon being at least partially housed within said inner member;
iii. a tampon positioning member being disposed within said inner member and co-axially with said tampon for expelling said tampon from said outer member, wherein said tampon positioning member is joined to said interior surface of said inner member between said first end and said second end; and
wherein a portion of said tampon remains in said inner member when said inner member is fully engaged with said outer member to define a remaining portion of said tampon, said remaining portion of said tampon requiring a force of less than about 0.30 N to be removed from said inner member.

6. The tampon applicator of claim 5 wherein said remaining portion of said tampon requires a force of less than about 0.20 N to be removed from said inner member.

7. The tampon applicator of claim 5 wherein said remaining portion of said tampon requires a force of less than about 0.15 N to be removed from said inner member.

8. The tampon applicator of claim 5 wherein said first end of said inner member extends beyond said insertion end of said outer member when said inner member is fully engaged with said outer member.

9. The tampon applicator of claim 5 wherein said tampon remains in contact with said tampon positioning member when said inner member is fully engaged with said outer member.

10. The tampon applicator of claim 5 wherein said outer member conforms to a profile of said tampon.

11. The tampon applicator of claim 5 further comprising an annular ring located on said exterior surface of said outer member.

12. A tampon applicator for positioning a tampon inside a vaginal cavity, said applicator comprising:
i. an outer member disposed co-axially with an inner member for insertion of said tampon into said vaginal cavity, said inner member being slidable within said outer member, said outer member comprising an insertion end, an interior surface and an exterior surface, said inner member comprising an interior surface, an exterior surface, a first end and a second end;
ii. said tampon being at least partially housed within said inner member;
iii. a tampon positioning member being disposed within said inner member and co-axially with said tampon for expelling said tampon from said outer member, wherein said tampon positioning member is joined to said interior surface of said inner member between said first end and said second end; and
wherein a portion of said tampon remains in said inner member when said inner member is fully engaged with said outer member by pushing said inner member into said outer member to define a remaining portion of said tampon, said remaining portion of said tampon requiring a force of less than about 0.30 N to be removed from said tampon applicator.

13. The tampon applicator of claim 12 wherein said remaining portion of said tampon requires a force of less than about 0.20 N to be removed from said tampon applicator.

14. The tampon applicator of claim 12 wherein said remaining portion of said tampon requires a force of less than about 0.15 N to be removed from said tampon applicator.

15. The tampon applicator of claim 12 wherein said first end of said inner member extends beyond said insertion end of said outer member when said inner member is fully engaged with said outer member.

16. The tampon applicator of claim 12 wherein said tampon remains in contact with said tampon positioning member when said inner member is fully engaged with said outer member.

17. The tampon applicator of claim 12 wherein said outer member conforms to a profile of said tampon.

18. The tampon applicator of claim 12 further comprising an annular ring located on said exterior surface of said outer member.
